(19) United States
(12) Patent Application Publication
(10) Pub. No.: US 2017/0100569 A1
(43) Pub. Date: Apr. 13, 2017

(54) TRIGGERED RELEASE MECHANISM TO IMPROVE EFFICACY OF DRUG COATED BALLOONS

(71) Applicant: The Spectranetics Corporation, Colorado Springs, CO (US)

(72) Inventor: Blaine Schneider, Colorado Springs, CO (US)

(21) Appl. No.: 15/282,579

(22) Filed: Sep. 30, 2016

Related U.S. Application Data
(60) Provisional application No. 62/239,121, filed on Oct. 8, 2015.

(51) Int. Cl. A61M 25/10; A61L 29/16; A61L 29/08; A61L 29/14

(52) U.S. Cl. CPC A61M 25/104; A61L 29/14; A61L 29/16; A61L 29/085; A61L 2420/06; A61L 2300/416; A61M 2025/105

(57) ABSTRACT

A method for treating a target vascular portion of a subject including: providing an angioplasty balloon system including a balloon, the balloon carrying a first polymer, a second polymer, and at least one active agent on a surface of the balloon, the at least one active agent being at least partially contained within at least one of the first polymer and the second polymer, wherein the first polymer and the second polymer having different stereoisoisomeric forms that can co-crystalize to form a stereocomplex between the first and second polymers; positioning the balloon proximate the target vascular portion; expanding the balloon to engage the target vascular portion; thereby delivering at least a portion of the active agent to the target vascular portion; and withdrawing the balloon from the subject.

### CROSS REFERENCE TO RELATED APPLICATION

The present application claims the benefit of and priority to, under 35 U.S.C. §119(e), U.S. Provisional Application Ser. No. 62/239,121, filed Oct. 8, 2015, entitled TRIGGERED RELEASE MECHANISM TO IMPROVE EFFICACY OF DRUG COATED BALLOONS. The above application is hereby incorporated by reference in its entirety for all that they teach and for all purposes.

### FIELD OF THE DISCLOSURE

The present disclosure relates generally to the use of medical devices for the treatment of vascular conditions. In particular, the present disclosure provides methods for treating vascular stenoses by delivering therapeutic agents via balloon catheters to the stenoses.

### BACKGROUND

Coronary artery disease (CAD) is a common form of heart disease, affecting millions of Americans. Peripheral artery disease (PAD), also called peripheral arterial disease, is a common circulatory problem in which narrowed arteries reduce blood flow in an individual's limbs, such as arms and legs. CAD and PAD often result from a condition known as atherosclerosis, which generally manifests as the accumulation of a waxy substance on the inside of a subject's arteries. This substance, called plaque, is made of cholesterol, fatty compounds, calcium, and a blood-clotting material called fibrin. As the plaque builds up, the artery narrows, or becomes stenotic, making it more difficult for blood to flow to the heart and/or extermities.

Balloon angioplasty and other transluminal medical treatments are well-known and have been proven efficacious in the treatment of stenotic lesions at the core of CAD and PAD. In a typical angioplasty procedure, a catheter is inserted into the groin or arm of a subject and guided forward into, in the case of CAD, the coronary arteries of the heart or, in the case of PAD, into the narrowed portion of the arteries in the legs. There, blocked (partially blocked or fully blocked) arteries can be unblocked by increasing the size of the passageway within the artery with a balloon positioned at the tip of the catheter. Initially, angioplasty was performed only with balloon catheters, but technical advances have been made and improved patient outcomes have been achieved with the placement of small metallic spring-like devices called “stents” at the site of the blockage. The implanted stent serves as a scaffold that keeps the artery open. Angioplasty and stenting techniques are widely used around the world and provide an alternative option to bypass surgery for improving blood flow to the heart muscle. There are, however, limitations associated with angioplasty and stenting, one of which is called “restenosis.”

Restenosis occurs when the treated vessel becomes blocked again—when the stenosis reforms within the vessel. For example, when a stent is placed in a blood vessel, new tissue grows inside the stent, covering the struts of the stent. Initially, this new tissue consists of healthy cells from the lining of the arterial wall (that is, endothelium). This is a favorable effect because development of normal lining over the stent allows blood to flow smoothly over the stented area without clotting. Later, scar tissue may form underneath the new healthy lining. However, in about 25 percent of patients, the growth of scar tissue underneath the lining of the artery may be so thick that it can obstruct the blood flow and produce another blockage. “In-stent” restenosis is typically seen 3 to 6 months after the initial procedure. Another significant limitation of the use of stents is stent thrombosis, which, although rare (occurring in only 1 percent of patients), most commonly presents as acute myocardial infarction.

In addition to angioplasty and the deployment of stents, other types of intervention for stenotic vessels include atherectomy, bypass surgery, and the use of laser ablation and mechanical cutting systems to reduce the plaque size. Treatments using various pharmacological agents have also been developed, including drug-coated balloons (DCB). Exemplary drug-coated balloons are disclosed in U.S. Pat. No. 9,011,896, the disclosures of which are hereby incorporated by reference in their entirety.

In a DCB, a drug is provided, sometimes with a coating or other substance, on an outer surface of the balloon. When the balloon is inflated, the drug-coated surface is placed into contact with the passageway within the artery.

However, in a typical drug-coated balloon, a portion of the drug is not taken up by the artery wall at the point of inflation. According to some studies, about 10% or more of the initial drug dosage is lost during transport to the inflation point, about 80% of the initial drug dosage is released upon inflation, and about 10% of the initial drug dosage remains on the balloon at the end of the procedure. Of the released drug dosage, only about 20% is taken up by the vessel wall, while the rest is washed of distally. In some cases, a majority of the drug may be delivered in an active form to locations other than the intended target.

Improvements in one or more aspects of the foregoing are desired.

### SUMMARY

Given the persistence of CAD and PAD, the most efficacious means for improving therapeutic outcomes may involve combinations of therapies designed not only to reduce plaque size in the short term, but also to prevent future complications such as restenosis. These and other needs are addressed by the various aspects, embodiments, and configurations of the present disclosure.

Apparatus and methods according to the present disclosure generally relate to treating vascular stenoses (for example, scar tissue, plaque build-up, calcium deposits and other types of undesirable lesion) by using balloon angioplasty and drug delivery via drug-coated balloons.

In one exemplary embodiment, an angioplasty balloon is provided. The angioplasty balloon comprises an inflatable balloon including a balloon surface, and a coating on at least a portion of the balloon surface. The coating includes a therapeutic agent, a first polymer, and a second polymer. The first polymer and the second polymer having different stereoisoisomeric forms, wherein the first and the second polymer can co-crystalize to form a stereocomplex between the first and second polymers.

In a more particular embodiment of the above embodiment, the therapeutic agent comprises one or more substances selected from the group consisting of: an antiproliferative agent, an antimitotic agent, an antiplatelet agent, an alkylating agent, an antimetabolite, a platinum coordination complex, a hormone, an anticoagulant, a fibrinolytic agent, an antimigratory agent, an antisecretory agent, an anti-inflammatory agent, indole and acetic acids, an immunosuppressive agent, an angiogenic agent, an angiotensin receptor blocker, a nitric oxide donor, an anti-sense oligonucleotide, a cell cycle inhibitor, a retinoid, a cyclin/CDK inhibitor, an HMG co-enzyme reductase inhibitor, and a protease inhibitor. In another more particular embodiment of any of the above embodiments, the therapeutic agent comprises at least one antiproliferative agent. In still another more particular embodiment of any of the above embodiments, the therapeutic agent comprises a restenosis inhibitor. In yet still another more particular embodiment of any of the above embodiments, the therapeutic agent comprises paclitaxel.

In a more particular embodiment of any of the above embodiments, the first polymer comprises a first nanoparticle and the second polymer comprises a second nanoparticle. In a more particular embodiment of any of the above embodiments, the therapeutic agent is at least partially contained within at least one of the first nanoparticle and the second nanoparticle.

In a more particular embodiment of any of the above embodiments, the first polymer and second polymer each comprise a polylactide. In another more particular embodiment of any of the above embodiments, the first polymer comprises poly(L-lactide) and the second polymer comprises poly(D-lacticde). In still another more particular embodiment of any of the above embodiments, the first polymer comprises isotatic poly(L-lactide) and the second polymer comprises isotatic poly(D-lacticde).

In another exemplary embodiment, a method for treating a target vascular portion of a subject is provided. The method includes providing an angioplasty balloon system including a balloon, the balloon carrying a first polymer, a second polymer, and at least one active agent on a surface of the balloon. The at least one active agent is at least partially contained within at least one of the first polymer and the second polymer. The first polymer and the second polymer have different stereoisoisomeric forms that can co-crystalize to form a stereocomplex between the first and second polymers. The method further includes positioning the balloon proximate the target vascular portion. The balloon is then expanded to engage the target vascular portion, thereby delivering at least a portion of the active agent to the target vascular portion. The balloon is then withdrawn from the subject.

In a more particular embodiment, the method also includes forming a stereocomplex between the first and second polymers. Formation of the stereocomplex releases the active agent from the at least one of the first polymer and second polymer. In a still more particular embodiment, the stereocomplex is formed within one or more cells comprising the target vascular portion, whereby the formation of the stereocomplex releases a therapeutically effective amount of the therapeutic agent in the one or more cells.

In a more particular embodiment of any of the above methods, the first polymer comprises a first nanoparticle and the second polymer comprises a second nanoparticle. In a still more particular embodiment of any of the above methods, the stereocomplex is formed by the first and the second polymers being driven together to form a third particle by stereocomplexation.

In a more particular embodiment of any of the above methods, the therapeutic agent comprises one or more substances selected from the group consisting of: an antiproliferative agent, an antimitotic agent, an antiplatelet agent, an alkylating agent, an antimetabolite, a platinum coordination complex, a hormone, an anticoagulant, a fibrinolytic agent, an antimigratory agent, an antisecretory agent, an anti-inflammatory agent, indole and acetic acids, an immunosuppressive agent, an angiogenic agent, an angiotensin receptor blocker, a nitric oxide donor, an anti-sense oligonucleotide, a cell cycle inhibitor, a retinoid, a cyclin/CDK inhibitor, an HMG co-enzyme reductase inhibitor, and a protease inhibitor. In a still more particular embodiment, the therapeutic agent comprises a restenosis inhibitor. In a still yet more particular embodiment, the therapeutic agent comprises paclitaxel.

In a more particular embodiment of any of the above methods, the first polymer and second polymer each comprise a polylactide. In a still more particular embodiment, the first polymer comprises poly(L-lactide) and the second polymer particle comprises poly(D-lacticde).

In one exemplary embodiment, an angioplasty balloon is provided. The angioplasty balloon comprises an inflatable balloon including a balloon surface; and a coating on at least a portion of the balloon surface. The coating includes a polymersome enclosing a therapeutic agent.

In a more particular embodiment, the polymersome comprises a polymeric membrane and at least one DNA nanopore, the DNA nanopore providing a pathway through the polymeric membrane. In an even more particular embodiment, the pathway defines a lumen diameter of about 2 nm.

In a more particular embodiment of any of the above embodiments, the polymersome comprises an amphiphilic block copolymer, and even more particularly comprises poly 2-(methacryloyloxy)ethyl phosphorylcholine-b-disisopropylamino) ethyl methacrylate.

In a more particular embodiment of any of the above embodiments, the therapeutic agent comprises one or more substances selected from the group consisting of: an antiproliferative agent, an antimitotic agent, an antiplatelet agent, an alkylating agent, an antimetabolite, a platinum coordination complex, a hormone, an anticoagulant, a fibrinolytic agent, an antimigratory agent, an antisecretory agent, an anti-inflammatory agent, indole and acetic acids, an immunosuppressive agent, an angiogenic agent, an angiotensin receptor blocker, a nitric oxide donor, an anti-sense oligonucleotide, a cell cycle inhibitor, a retinoid, a cyclin/CDK inhibitor, an HMG co-enzyme reductase inhibitor, and a protease inhibitor. In a still more particular embodiment, the therapeutic agent comprises a restenosis inhibitor. In a still yet more particular embodiment, the therapeutic agent comprises paclitaxel.

In another exemplary embodiment, a method for treating a target vascular portion of a subject is provided. The method includes providing an angioplasty balloon system including a balloon and a coating on at least a portion of an outer surface of the balloon. The coating includes a polymersome enclosing a therapeutic agent. The method further includes positioning the balloon proximate the target vascular portion. The balloon is then expanded to engage the target vascular portion, thereby delivering at least a portion of the active agent to the target vascular portion. The balloon is then withdrawn from the subject.

In a more particular embodiment of any of the above methods, the polymersome comprises a polymeric membrane and at least one DNA nanopore, the DNA nanopore providing a pathway through the polymeric membrane. In one more particular embodiment, the pathway defines a lumen diameter of about 2 nm. In another more particular embodiment, the therapeutic agent is encapsulated by the polymeric membrane, and delivering the portion of the active agent includes releasing the therapeutic agent from inside the polymersome through the DNA nanopore, where it is absorbed by a cell in the target vascular portion.

In a more particular embodiment, the method also includes forming a stereocomplex between the first and second polymers. Formation of the stereocomplex releases the active agent from the at least one of the first polymer and second polymer. In a still more particular embodiment, the stereocomplex is formed within one or more cells comprising the target vascular portion, whereby the formation of the stereocomplex releases a therapeutically effective amount of the therapeutic agent in the one or more cells.

In a more particular embodiment of any of the above embodiments, the polymersome comprises an amphiphilic block copolymer, and even more particularly comprises poly 2-(methacryloyloxy)ethyl phosphorylcholine-b-disisopropylamino) ethyl methacrylate.

In a more particular embodiment of any of the above methods, the therapeutic agent comprises one or more substances selected from the group consisting of: an antiproliferative agent, an antimitotic agent, an antiplatelet agent, an alkylating agent, an antimetabolite, a platinum coordination complex, a hormone, an anticoagulant, a fibrinolytic agent, an antimigratory agent, an antisecretory agent, an anti-inflammatory agent, indole and acetic acids, an immunosuppressive agent, an angiogenic agent, an angiotensin receptor blocker, a nitric oxide donor, an anti-sense oligonucleotide, a cell cycle inhibitor, a retinoid, a cyclin/CDK inhibitor, an HMG co-enzyme reductase inhibitor, and a protease inhibitor. In a still more particular embodiment, the therapeutic agent comprises a restenosis inhibitor. In a still yet more particular embodiment, the therapeutic agent comprises paclitaxel.

These and other advantages will be apparent from the disclosure of the aspects, embodiments, and configurations contained herein.

As used herein, “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (for example, X₁ and X₂) as well as a combination of elements selected from two or more classes (for example, Y₁ and Zₒ).

It is to be noted that the term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “amphiphilic” as used herein generally refers to a material that is at least partially dissolvable in aqueous solvents, such as blood in-vivo, as well as at least partially dissolvable in non-aqueous solvents, such as ethanol, methanol, and/or isopropanol.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material”.

The terms “vasculature” and “vascular” as used herein refer to any part of the circulatory system of a subject, including peripheral and non-peripheral arteries and veins. Vascular material found within the vasculature can be comprised of both biological material (for example, nucleic acids, amino acids, carbohydrates, polysaccharides, lipids and the like) and non-biological material (for example, fat deposits, fibrous tissue, calcium deposits, remnants of dead cells, cellular debris and the like).

A “catheter” is a tube that can be inserted into a body cavity, duct, lumen, or vessel, such as the vasculature system. In most uses, a catheter is a relatively thin, flexible tube (“soft” catheter), though in some uses, it may be a larger, solid-less flexible—but possibly still flexible—catheter (“hard” catheter).

The term “balloon catheter” as used herein generally refers to the various types of angioplasty catheters which carry a balloon for performing angioplasty. Balloon catheters may also be of a wide variety of inner structure, such as different lumen design, of which there are at least three basic types: triple lumen, dual lumen and co-axial lumen. All varieties of internal structure and design variation are meant to be included by use of the term “balloon catheter” herein.

The term “stenosis” as used herein generally refers to an abnormal narrowing in a blood vessel, the vasculature or other tubular organ or structure. There are many causes of a stenosis. One cause is atherosclerosis (also known as arteriosclerotic vascular disease), which is a specific form of arteriosclerosis in which a vasculature wall thickens as a result of invasion and accumulation of white blood cells. The thickening of the wall can lead to the formation of a thrombus within the lumen of the vasculature, whereby the thrombus may fully or partially occlude the lumen. “Restenosis” is the recurrence of stenosis after a procedure to initially treat the stenosis.

The term “therapeutic agent” as used herein generally refers to any known or hereafter discovered pharmacologically active agent that provides therapy to a subject through the alleviation of one or more of the subject's physiological symptoms. A therapeutic agent may be a compound that occurs in nature, a chemically modified naturally occurring compound, or a compound that is chemically synthesized. The agent will typically be chosen from the generally recognized classes of pharmacologically active agents, including the following: analgesic agents; anesthetic agents; antiarthritic agents; respiratory drugs, including antiasthmatic agents; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; antiinflammatory agents; antimigraine preparations; antinauseants; antiparkinsonism drugs; antipruritics; antipsychotics; antipyretics; antispasmodics; antitubercular agents; antiulcer agents; antiviral agents; anxiolytics; appetite suppressants; attention deficit disorder (ADD) and attention deficit hyperactivity disorder (ADHD) drugs; cardiovascular preparations including calcium channel blockers, CNS agents; beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; restenosis inhibitors; muscle relaxants; narcotic antagonists; nicotine; nutritional agents, such as vitamins, essential amino acids and fatty acids; ophthalmic drugs such as antiglaucoma agents; parasympatholytics; psychostimulants; sedatives; steroids; sympathomimetics; tranquilizers; and vasodilators including general coronary, peripheral and cerebral.

The term “therapeutically effective amount” refers to a sufficient amount of the therapeutic agents to treat disorders, at a reasonable benefit/risk ratio applicable to any medical treatment. It will be understood, however, that the total daily usage of the therapeutic agents and compositions of embodiments of the invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the therapeutic agent at levels lower than required to achieve the desired therapeutic effect and to gradually increase the dosage until the desired effect is achieved.

The term “means” as used herein shall be given its broadest possible interpretation in accordance with 35 U.S.C., Section 112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

It should be understood that every maximum numerical limitation given throughout the present disclosure is deemed to include each and every lower numerical limitation as an alternative, as if such lower numerical limitations were expressly written herein. Every minimum numerical limitation given throughout the present disclosure is deemed to include each and every higher numerical limitation as an alternative, as if such higher numerical limitations were expressly written herein. Every numerical range given throughout the present disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a schematic view of an exemplary embodiment of a balloon system in accordance with the present disclosure; a balloon of a balloon catheter of the system is illustrated in an expanded configuration and a distally advanced position relative to a protective sheath;

FIG. 1B is a schematic view of another exemplary embodiment of a balloon system in accordance with the present disclosure without a protective sheath;

FIG. 2A is an elevation longitudinal section view of the balloon catheter of FIG. 1A longitudinally offset from a target to be treated; the balloon is in an unexpanded configuration and a proximally retracted position within the protective sheath;

FIG. 2B is an elevation longitudinal section view of the balloon catheter of FIG. 1A; the balloon is in a distally advanced position relative to the protective sheath and is longitudinally aligned with the target and radially offset from the target;

FIG. 2C is an elevation longitudinal section view of the balloon catheter of FIG. 1A; the balloon is in the distally advanced position relative to the protective sheath and is longitudinally aligned with the target and radially expanded to engage the target; and

FIG. 3 is a flow diagram of an exemplary method for treating vascular stenosis via a drug-coated balloon in accordance with the present disclosure.

### DETAILED DESCRIPTION

Methods according to the present disclosure generally relate to treating vascular stenoses (for example, scar tissue, plaque build-up, calcium deposits and other types of undesirable lesion) by using drug delivery via a balloon system. Generally and in some embodiments, the balloon system includes a drug-coated balloon (DCB) catheter, which is described in more detail below. The DCB catheter is inserted into and delivers one or more therapeutic agents to the vasculature of a subject. In some embodiments, the balloon system also includes an inflation fluid source that delivers an inflation fluid to the DCB catheter to cause the balloon of the DCB catheter to inflate or expand and, in some embodiments, deliver the therapeutic agent(s) to the vasculature.

An example of a DCB catheter includes the Stellarex™ drug coated angioplasty balloon (DCB) platform is designed to treat peripheral arterial disease. The Stellarex™ DCB platform uses EnduraCoat™ technology, a durable, uniform coating designed to prevent drug loss during transit and facilitate controlled, efficient drug delivery to the treatment site. The Stellarex™ DCB platform received CE mark to be marketed in the European Union in December 2014. At the time of filing this application, the Stellarex™ DCB platform is not approved in the United States.

Other examples of DCB catheters in accordance with the present disclosure include those available from Lutonix, Inc. of New Hope, Minn. under the tradename Lutonix®, such as the Lutonix® 014 catheter and those available from Medtronic PLC of Fridley, Minn. under the tradename IN.PACT®. Further examples of DCB catheters, therapeutic agents, and balloon coatings including therapeutic agents in accordance with the present disclosure include those disclosed in U.S. Pat. Nos. 8,114,429; 8,128,951; 8,257,304; 8,257,722; 8,491,925; 8,563,023; 8,673,332; 8,734,825, 8,740,841; 9,011,896; U.S. Pat. Apps. 62/098,242; 13/628,608; 13/707,401; 11/411,635; 60/680,450; 13/310,320; 12/712,134; 12/558,420; 12/210,344; 14/149,862; 13/560,538; 13/926,515; 61/665,758; 13/628,627; 13/975,209; 13/975,220; 13/975,228; 14/032,336; 14/162,900; 14/254,160; 14/731,715; the entireties of which are incorporated by reference herein for all purposes. Another example of a balloon-type catheter product to which a drug coating could be applied includes the AngioSculpt™ scoring balloon catheter, which is produced (without the drug coating) by AngioScore, Inc. of Colorado Springs, Colo., which is the applicant of the present patent application.

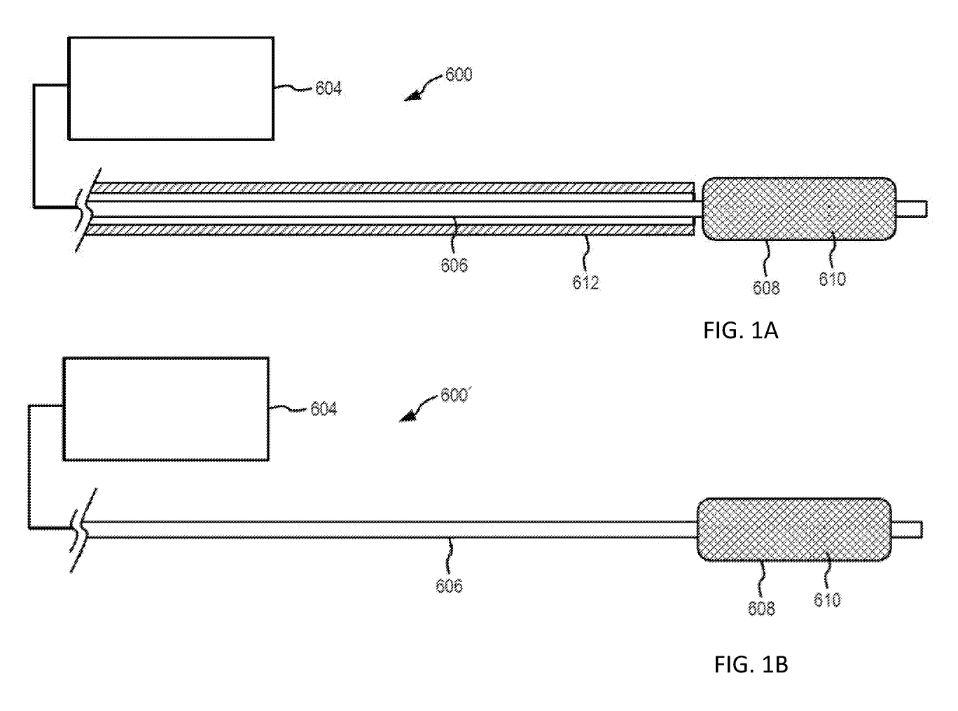

Referring now to FIG. 1A, an exemplary embodiment of a balloon system 600 in accordance with the present disclosure is illustrated. The balloon system 600 includes a DCB catheter 602 that receives inflation fluid from an inflation fluid source 604. The DCB catheter 602 includes a tubular element 606 that carries a drug-coated expandable element or balloon 608. The tubular element 606 includes an inflation lumen (not illustrated) that receives inflation fluid from the inflation fluid source 604 and delivers the inflation fluid to the balloon 608 to inflate the balloon 608. In some embodiments, the tubular element 606 also includes a guidewire lumen (not illustrated) for receiving a guidewire (not illustrated) to guide the DCB catheter 602 to the target.

The balloon 608 carries a coating 610 that includes at least one or more therapeutic agents, a first polymer, and a second polymer, as described in more detail below.

As shown in FIG. 1A, in some embodiments, the DCB catheter 620 further includes a protective sheath 612 that is translatable relative to the tubular element 606 and the balloon 608. The protective sheath 612 initially surrounds the unexpanded balloon 608 to prevent the coating 610 from prematurely dissolving when the DCB catheter 620 is inserted into the vasculature of the subject.

Referring next to FIG. 1B, another exemplary balloon system 600′ is illustrated. Balloon system 600′ is similar to balloon system 600, and similar parts are indicated by similar part numbers. As shown in FIG. 1B, exemplary balloon system 600′ does not include a protective sheath 612. In some more particular embodiments, an excipient, coating, or other suitable material is placed over at least a portion of the surface of balloon 608 to prevent all, most, or a substantial portion of the coating 610 from prematurely dissolving when the DCB catheter 620 is inserted into the vasculature of the subject.

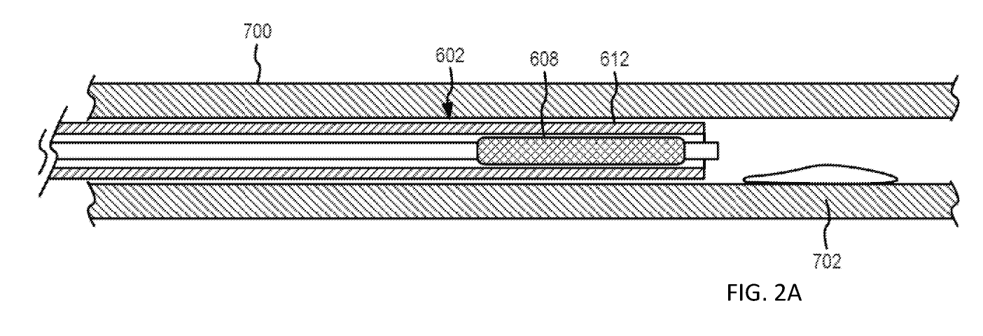

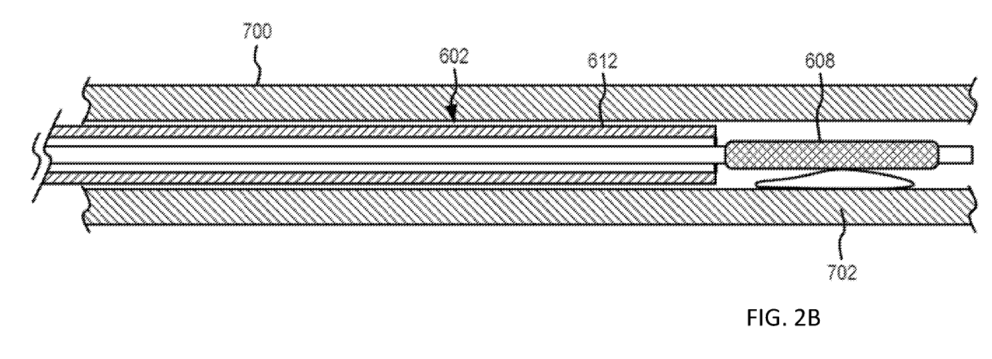

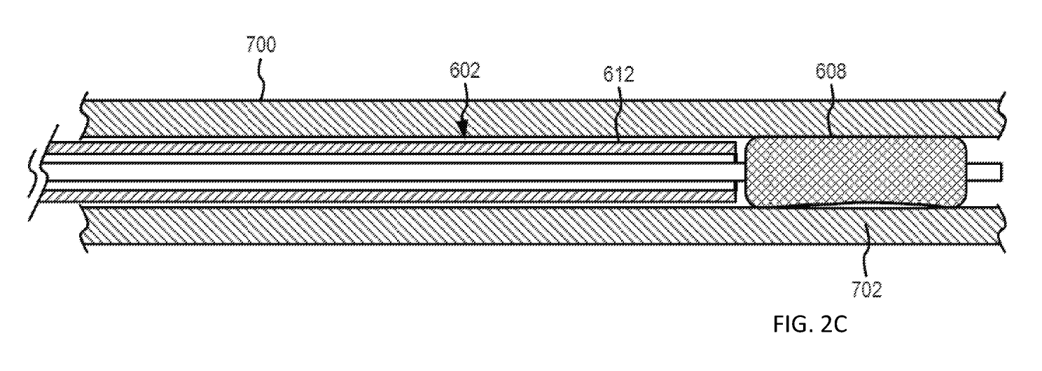

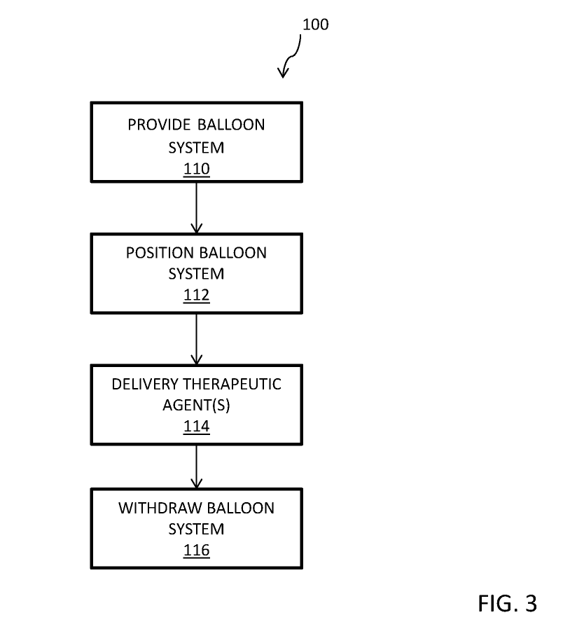

Referring next to FIGS. 2A-2C and FIG. 3, and exemplary method 100 for treatment of vascular stenosis by drug delivery via drug-coated balloons is provided. As shown in block 110, a balloon system is provided. In one exemplary embodiment, the balloon system is balloon system 600. In another exemplary embodiment, the balloon system is balloon system 600′.

In block 112, the balloon system 600 is positioned in an appropriate position for delivering the therapeutic agent(s) to the vasculature of the subject, as shown in FIG. 2A. In some embodiments, positioning the balloon system in an appropriate position includes (1) as illustrated in FIG. 2A, positioning the DCB catheter 602 in the vasculature 700 of the subject such that the catheter 602 is longitudinally offset (that is, offset in a longitudinal direction of the catheter 602) from the target 702 to be treated; the balloon 608 may be in an unexpanded configuration, and if the balloon system 600 includes a protective sheath 612, the balloon 608 may be in a proximally retracted position within the protective sheath 612; (2) as illustrated in FIG. 2B, translating the balloon 608 to a distally advanced position relative to the protective sheath 612 such that the balloon 608 is longitudinally aligned with the target 702 and radially offset (that is, offset in a radial direction of the catheter 602) from the target 702; and (3) as illustrated in FIG. 2C, expanding the balloon 608 in the radial direction to contact the target 702. If the balloon system 600 does not include a protective sheath 612, the balloon 608 is longitudinally aligned with the target 702 and radially offset (that is, offset in a radial direction of the catheter 602) from the target 702; and the balloon 608 is expanded in the radial direction to contact the target 702. In some embodiments, the inflation fluid source 604 delivers inflation fluid to the balloon 608 to expand the balloon 608.

At block 114, the balloon 608 delivers the therapeutic agent(s) to the target 702. In some embodiments, the balloon 608 delivers the therapeutic agent(s) to the target 702 by the balloon 608 contacting the blood of the subject, thereby dissolving the coating 610, and/or expanding the balloon 608 to contact the target 702.

The method concludes by withdrawing the balloon system from the subject, as shown at block 116. In some embodiments, withdrawing the balloon system from the subject includes removing the DCB catheter 602 from the vasculature 700 of the subject.

In one exemplary embodiment, the therapeutic agent is a substance useful for treating a vascular condition. Exemplary therapeutic agents include:

(1) antiproliferative and antimitotic agents such as natural products such as vinca alkaloids (i.e. vinblastine, vincristine, and vinorelbine), paclitaxel, epidipodophyllotoxins (i.e. etoposide, teniposide), antibiotics (dactinomycin, actinomycin D, daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine);

(2) antiplatelet agents such as G(GP) inhibitors and vitronectin receptor antagonists;

(3) alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC);

(4) antiproliferative and antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine});

(5) platinum coordination complexes such as cisplatin, carboplatin, procarbazine, hydroxyurea, mitotane, and aminoglutethimide;

(6) hormones (e.g. estrogen);

(7) anticoagulants (heparin, synthetic heparin salts and other inhibitors of thrombin);

(8) fibrinolytic agents (such as tissue plasminogen activator, streptokinase and urokinase), aspirin, dipyridamole, ticlopidine, clopidogrel, abciximab;

(9) antimigratory agents;

(10) antisecretory agents (breveldin);

(11) anti-inflammatory agents, such as adrenocortical steroids (cortisol, cortisone, fludrocortisone, prednisone, prednisolone, 6.alpha.-methylprednisolone, triamcinolone, betamethasone, and dexamethasone), non-steroidal agents (salicylic acid derivatives i.e. aspirin; para-aminophenol derivatives i.e. acetaminophen;

(12) indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate);

(13) immunosuppressive agents such as cyclosporine, tacrolimus (FK-506), sirolimus (rapamycin), azathioprine, mycophenolate, mofetil;

(14) angiogenic agents such as vascular endothelial growth factor (VEGF), fibroblast growth factor (FGF);

(15) angiotensin receptor blockers;

(16) nitric oxide donors;

(17) anti-sense oligionucleotides and combinations thereof;

(18) cell cycle inhibitors, mTOR inhibitors, and growth factor receptor signal transduction kinase inhibitors;

(19) retenoids;

(20) cyclin/CDK inhibitors;

(21) HMG co-enzyme reductase inhibitors (statins); and

(22) protease inhibitors.

Therapeutic agents in accordance with the present disclosure can be chosen based upon functional characteristics, including, but not necessarily limited to, the ability to inhibit restenosis, mitosis or cellular proliferation. In one exemplary embodiment, For example, a therapeutic agent can be a taxane, including paclitaxel, docetaxel, protaxel, DHA-paclitaxel, PG-paclitaxel, docosahexaenoic acid (DHA), or any combinations or derivatives thereof capable of inhibiting mitosis or cellular proliferation. In some cases, the presence of a mitotic inhibitor prevents restenosis that may occur in the absence of the inhibitor. Other examples of therapeutic agents include rapamycin (for example, sirolimus) or a derivative of rapamycin (for example, everolimus), or any combinations or derivatives thereof. Additionally or alternatively, specific inhibitors of neovascularization such as thalidomide, statins such as atorvastatin, cerivastatin, fluvastatin, or anti-inflammatory drugs like corticoids or lipophilic derivatives of corticoids such as betamethasone diproprionate or dexa-methasone-21-palmitate are examples of oxitherapeutic agents that can be used in accordance with the present disclosure. In some cases, the therapeutic agent is stable against oxidative degradation, or oxidation in sensitive. Various therapeutic agents may be applied or combined if different pharmacological actions are required or efficacy or tolerance is to be improved.

In one exemplary embodiment, the therapeutic agent is selected from the group consisting of paclitaxel, sirolimus, and gemcitabine. In are more particular embodiment, the therapeutic agent is selected from the group consisting of paclitaxel and sirolimus. In one more particular embodiment, the therapeutic agent is paclitaxel. In another more particular embodiment, the therapeutic agent is sirolimus. In still another more particular embodiment, the therapeutic agent is gemcitabine.

Coatings in accordance with some exemplary embodiments of the present disclosure include a therapeutic agent a first polymer, and a second polymer. The first polymer and second polymer are capable of co-crystalizing to form a sterocomplex between the first and second polymers. Exemplary pairs of first and second polymers are disclosed in L. Sun, et al., “Structural reorganization of cylindrical nanoparticles triggered by polylactide sterocomplexation,” Nature Communications, vol. 5, 5746, pages 1-9 and supplemental 1-21, 17 Dec. 2014, the disclosures of which are hereby incorporated by reference in their entirety.

Exemplary pairs of first and second polymers include:
poly(L-lactide)b-poly(acrylic acid) and poly(D-lactide)b-poly(acrylic acid);
poly(L-lactide)-b-poly(ethylene glycol) and poly(D-lactide)-b-poly(ethylene glycol);
poly(L-lactide)-b-poly(ethylene oxide) and poly(D-lactide)-b-poly(ethylene oxide);
polyethylene glycol-b-poly(L-lactide)-b-poly(L-lactide) and polyethylene glycol-b-poly(D-lactide)-b-poly(D-lactide); and
poly-(L-lactide)-b-poly(ε-caprolactam) and poly-(D-lactide)-b-poly(ε-caprolactam).

In some exemplary embodiments, the first polymer and the second polymers are provided in substantially cylindrical form. In a more particular embodiment, the first polymer and the second polymers form stereocomplex micelles. In some embodiments, the therapeutic agent is at least partially encapsulated by or contained within the cylinders of the first polymer and/or the cylinders of the second polymer. Upon stereocomplexation to form the miscelles, the therapeutic agent is released from the first polymer and/or second polymer.

In a more particular embodiment, the first polymer and the second polymer are present as nanoparticles. In a more particular embodiment, each particle has at least one dimension measuring 100 nm or less. In an even more particular embodiment, each particle has at least one dimension as little as 1 nm, 5 nm, 10 nm, 25 nm, as great as 50 nm, 75 nm, or 100 nm, or within any range defined between any two of the foregoing values, such as 1 nm to 100 nm.

Illustratively, when the first and second polymer nanoparticles are in close enough proximity, the polymer chains of the first polymer and the second nanoparticles are a driven together to form a stereocomplex, a third particle, through the process of stereocomplexation.

Complexation refers to the mixing of two polymers leading to the self-assembly of a complex, whose properties may be very different from those of the parent polymers. Complexation can occur between polymers with different chemical natures, tacticities (i.e. between an isotactic and a syndiotactic polymer) or chiralities (two isotactic polymers of different configurations). Complexation between tacticities or chiralities is known as stereocomplexation.

Without wishing to be held to any particular theory, it is believed that stereocomplexation would be unable to occur under the conditions present on the surface of the blood coated balloon. In addition, when in the blood stream the first and second particles would be unable to interact sufficiently to form the sterocomplex, and the therapeutic agent would not be released from the nanoparticles into the blood stream. In contrast, when both types of polymers are taken into a cell, such as a cell at the point of inflation of the drug-coated balloon, the release of the therapeutic through stereocomplxation would occur. Without wishing to be held by any particular theory, cellular process such as endocytosis, receptor-mediated endocytosis, membrane dialysis, translocation, active transport and direct penetration may all transport these particles into cells at the target site. In addition, active process such as sonophoresis or electrophoresis may be used to enhance the cellular uptake of the nanoparticles at the target site. As the concentration of both the first and second polymers decrease proximally through the blood stream, the ability of the two polymers to combine and release the therapeutic agent is reduced as the distance from the target vascular portion increases.

In some exemplary embodiments, the amount of bio-available therapeutic agent absorbed at or near the target vascular portion, based on the total amount of bio-available therapeutic agent, is as low as 10%, 20%, 25%, 30%, 40%, 50%, 60%, as high as 70%, 75%, 80%, 90%, 95%, 99%, or higher, or within any range defined between any two of the foregoing values, such as 10% to 99%, 25% to 95%, or 50% to 90%. In some exemplary embodiments, the total amount of bio-available therapeutic agent includes that portion of the therapeutic agent that has been released from the first polymer and/or second polymer and does not include that portion that has not been released from the first polymer or second polymer and is not bio-available.

In some exemplary embodiments, the therapeutic agent, first polymer, and second polymer are dispersed throughout a polymer matrix. The polymer coating may include additional components such as a plasticizer and/or wax. The therapeutic agent can be either water-soluble or water-insoluble. The polymer matrix may be complexed with iodine, or non-covalently bound iodine may be dispersed throughout the polymer matrix. In some embodiments, the polymer matrix is a non-ionic thermoplastic polymer or co-polymer. In some embodiments, the amphiphilic polymer is hydroxypropyl cellulose (HPC), polyvinyl pyrrolidone (PVP), polyethylene glycol (PEG), methyl cellulose, hydroxypropyl methylcellulose, or co-polymers of N-vinylpyrrolidone with other reactive double bond containing monomers such as styrene, acrylic acid, vinyl acetate or vinyl caprolactam. PVP and HPC exhibit higher solubility rates in aqueous solvents than PEG. Molecular weight of the polymers may also factor into solubility rates. In some embodiments, the PEG has as molecular weight of 1.5 KD to 50 KD. Co-polymers can be block or random.

Coatings in accordance with the present disclosure include an amphiphilic polymer coating that includes one or more therapeutic agents and one or more amphiphilic polymers or co-polymers. The amphiphilic polymer coating may include additional components such as a plasticizer and/or wax. The therapeutic agent can be either water-soluble or water-insoluble. Hydration of the amphiphilic polymer coating occurs immediately when exposed to aqueous fluids, such as blood in vivo, causing the amphiphilic polymer coating to dissolve and the therapeutic agent to release into tissue of the vasculature of the subject. Thus, the amphiphilic polymer coating is bioerodable in the sense that it is removable by bodily fluids, and non-durable. In some embodiments, the amphiphilic polymer or co-polymer is a non-ionic thermoplastic polymer or co-polymer. In some embodiments, the amphiphilic polymer is hydroxypropyl cellulose (HPC), polyvinyl pyrrolidone (PVP), polyethylene glycol (PEG), methyl cellulose, hydroxypropyl methylcellulose, or co-polymers of N-vinylpyrrolidone with other reactive double bond containing monomers such as styrene, acrylic acid, vinyl acetate or vinyl caprolactam. PVP and HPC exhibit higher solubility rates in aqueous solvents than PEG. Molecular weight of the polymers may also factor into solubility rates. In some embodiments, the PEG has as molecular weight of 1.5 KD to 50 KD. In some embodiments, the coating includes paclitaxel in PEG complexed with iodine in a polymer matrix, or non-covalently bound iodine may be dispersed throughout the polymer matrix. The PEG has a number average molecular weight, Mn, of about 8 KD. The amphiphilic polymer may also be a poly(hydroxyethyl methacrylic) acid, also known as poly(HEMA). In some embodiments, the poly(HEMA) has a number average molecular weight, Mn, below approximately 8 KD. In some embodiments, the poly(HEMA) has a number average molecular weight, Mn, of approximately 7 KD. In some embodiments, the amphiphilic polymer may be a co-polymer of HEMA with a monomer such as glycidyl methacrylate (GMA) or acrylic acid. Co-polymers can be block or random.

Coatings in accordance with the present disclosure may include various adjuvants and excipients to enhance efficacy or delivery of the therapeutic agents. For example, the therapeutic agents can be combined with lipophilic antioxidant such as nordihydroguaiaretic acid, resveratrol, propyl gallate, hydroxytoluene, butylated hydroxyanisole, and ascorbyl palmitate to enhance the adhesion of the therapeutic to the balloon 608. In some embodiments, the combination of a therapeutic agent such as paclitaxel and a lipophilic antioxidant such as nordihydroguaiaretic acid can be applied to the balloon 608 without the need for additional polymers.

Coatings in accordance with the present disclosure may be applied to balloons by using a variety of processes. For example, coatings may be applied to balloons using an automated coating apparatus, by dipping, sputtering, and hand coating. In one exemplary embodiment, the therapeutic agent is encapsulated in the nanoparticles prior to application of the coating on the balloon.

Coatings in accordance with other exemplary embodiments of the present disclosure include a polymersome. The polymersome is a small hollow sphere formed of a polymeric membrane. The polymeric membrane can be used to encapsulate a therapeutic agent. Exemplary polymersomes include nanocontainers as disclosed in L. Messager, et al., “Biomimetic Hybrid Nanocontainers with Selective Permeability,” Angew. Chem. Intl. Ed., vol. 55, pages 11106-11109 and supplemental 1-26, 25 Aug. 2016, the disclosures of which are hereby incorporated by reference in their entirety

In some exemplary embodiments, the polymeric membrane comprises one or more amphiphilic block copolymers, such as the amphiphilic bloc copolymer poly 2-(methacryloyloxy)ethyl phosphorylcholine-b-disisopropylamino) ethyl methacrylate (PMPC-b-PDPA). Exemplary PMPC-b-PDPA copolymers include PMPC₂₅-b-PDPA₇₂; PDPA₇₀-PMPC₂₅-S-S-PMPC₂₅-PDPA₇₀, and Cy3-labeled PMPC₂₅-PDPA₇₀, and mixtures thereof, such as a 95:5 ratio mixture of PMPC₂₅-PDPA₇₂: Cy3-PMPC₂₅-PDPA₇₂.

The polymersomes may be formed by suitable means such as self-assembly by thin-film hydration. In some exemplary embodiments, the polymersomes have a hydrodynamic diameter as little as 80 nm, 100 nm, 120 nm, as great as 150 nm, 180 nm, 200 nm, 250 nm, or within any range defined between any two of the forgoing values, such as 80 nm to 250 nm or 100 nm to 200 nm, for example. In some exemplary embodiments, the polymersomes have a polydispersity index of about 0.15. In some exemplary embodiments, the polymersomes membrane thickness as little as 5 nm, 5.3 nm, 6 nm, as great as 6.5 nm, 7 nm, 7.7 nm, 8 nm, or within any range defined between any two of the forgoing values, such as 5 nm to 8 nm or 5.3 nm to 7.7 nm, for example.

The therapeutic agent is illustratively encapsulated into the polymersome through electroporation of the polysmersome.

The polymersome illustratively includes one or more nanopores defining a lumen, the lumen providing an opening through the polymeric membrane. Exemplary nanopores include DNA nanopores and may have a lumen diameter of about 2 nm. In some exemplary embodiments, the DNA nanopores are inserted into the polymeric membrane following encapsulation of the therapeutic agent through a suitable process, such as incubation.

Without wishing to be held to any particular theory, it is believed that DNA nanopores provide a pathway through which the therapeutic agent can be transported out of the polymersome and into the blood stream or blood vessel wall at the target vascular site.

In some exemplary embodiments, the amount of bio-available therapeutic agent absorbed at or near the target vascular portion, based on the total amount of bio-available therapeutic agent, is as low as 10%, 20%, 25%, 30%, 40%, 50%, 60%, as high as 70%, 75%, 80%, 90%, 95%, 99%, or higher, or within any range defined between any two of the foregoing values, such as 10% to 99%, 25% to 95%, or 50% to 90%. In some exemplary embodiments, the total amount of bio-available therapeutic agent includes that portion of the therapeutic agent that has been released from the first polymer and/or second polymer and does not include that portion that has not been released from the first polymer or second polymer and is not bio-available.

In some exemplary embodiments, the polymersomes are dispersed throughout a polymer matrix. The polymer coating may include additional components such as a plasticizer and/or wax. The therapeutic agent can be either water-soluble or water-insoluble. The polymer matrix may be complexed with iodine, or non-covalently bound iodine may be dispersed throughout the polymer matrix. In some embodiments, the polymer matrix is a non-ionic thermoplastic polymer or co-polymer. In some embodiments, the amphiphilic polymer is hydroxypropyl cellulose (HPC), polyvinyl pyrrolidone (PVP), polyethylene glycol (PEG), methyl cellulose, hydroxypropyl methylcellulose, or co-polymers of N-vinylpyrrolidone with other reactive double bond containing monomers such as styrene, acrylic acid, vinyl acetate or vinyl caprolactam. PVP and HPC exhibit higher solubility rates in aqueous solvents than PEG. Molecular weight of the polymers may also factor into solubility rates. In some embodiments, the PEG has as molecular weight of 1.5 KD to 50 KD. Co-polymers can be block or random.

Coatings in accordance with the present disclosure include an amphiphilic polymer coating that includes one or more therapeutic agents and one or more amphiphilic polymers or co-polymers. The amphiphilic polymer coating may include additional components such as a plasticizer and/or wax. The therapeutic agent can be either water-soluble or water-insoluble. Hydration of the amphiphilic polymer coating occurs immediately when exposed to aqueous fluids, such as blood in vivo, causing the amphiphilic polymer coating to dissolve and the therapeutic agent to release into tissue of the vasculature of the subject. Thus, the amphiphilic polymer coating is bioerodable in the sense that it is removable by bodily fluids, and non-durable. In some embodiments, the amphiphilic polymer or co-polymer is a non-ionic thermoplastic polymer or co-polymer. In some embodiments, the amphiphilic polymer is hydroxypropyl cellulose (HPC), polyvinyl pyrrolidone (PVP), polyethylene glycol (PEG), methyl cellulose, hydroxypropyl methylcellulose, or co-polymers of N-vinylpyrrolidone with other reactive double bond containing monomers such as styrene, acrylic acid, vinyl acetate or vinyl caprolactam. PVP and HPC exhibit higher solubility rates in aqueous solvents than PEG. Molecular weight of the polymers may also factor into solubility rates. In some embodiments, the PEG has as molecular weight of 1.5 KD to 50 KD. In some embodiments, the coating includes paclitaxel in PEG complexed with iodine in a polymer matrix, or non-covalently bound iodine may be dispersed throughout the polymer matrix. The PEG has a number average molecular weight, Mn, of about 8 KD. The amphiphilic polymer may also be a poly(hydroxyethyl methacrylic) acid, also known as poly(HEMA). In some embodiments, the poly(HEMA) has a number average molecular weight, Mn, below approximately 8 KD. In some embodiments, the poly(HEMA) has a number average molecular weight, Mn, of approximately 7 KD. In some embodiments, the amphiphilic polymer may be a co-polymer of HEMA with a monomer such as glycidyl methacrylate (GMA) or acrylic acid. Co-polymers can be block or random.

Coatings in accordance with the present disclosure may include various adjuvants and excipients to enhance efficacy or delivery of the therapeutic agents. For example, the therapeutic agents can be combined with lipophilic antioxidant such as nordihydroguaiaretic acid, resveratrol, propyl gallate, hydroxytoluene, butylated hydroxyanisole, and ascorbyl palmitate to enhance the adhesion of the therapeutic to the balloon 608. In some embodiments, the combination of a therapeutic agent such as paclitaxel and a lipophilic antioxidant such as nordihydroguaiaretic acid can be applied to the balloon 608 without the need for additional polymers.

Coatings in accordance with the present disclosure may be applied to balloons by using a variety of processes. For example, coatings may be applied to balloons using an automated coating apparatus, by dipping, sputtering, and hand coating. In one exemplary embodiment, the therapeutic agent is encapsulated in the nanoparticles prior to application of the coating on the balloon.

### Prophetic Example #1

The balloon of a balloon catheter will be coated with a coating using an automated coating apparatus. The coating will include nanoparticles of isotatic poly(L-lactide) and isotatic poly(D-lacticde) having at least one dimension from about 1 nm to about 100 nm, wherein paclitaxel is encapsulated in at least one of the isotatic poly(L-lactide) nanoparticle and the isotatic poly(D-lacticde) nanoparticles. The coated balloon catheter will be positioned in the vasculature of a patient and expanded at a target position. The isotatic poly(L-lactide) and isotatic poly(D-lacticde) nanoparticles will be taken into cells of the vasculature at the target position, wherein the polymer chains of the isotatic poly(L-lactide) and isotatic poly(D-lacticde) nanoparticles will form a stereocomplex. The formation of the stereocomplex will release the paclitaxel in the interior of the cell.

### Prophetic Example #2

The balloon of a balloon catheter will be coated with a coating using an automated coating apparatus. The coating will include polymersomes having a polymeric membrane of the amphiphilic copolymer PMPC₂₅-b-PDPA₇₂ and membrane-spanning nanopores. The polymersomes will have a hydrodynamic diameter between about 100 and about 200 nm, and the nanopores will be DNA nanopores having out dimensions of 9 nm×6 nm and a lumen diameter of 2 nm. Paclitaxel is encapsulated in the polymersomes by the polymeric membrane. The coated balloon catheter will be positioned in the vasculature of a patient and expanded at a target position. The paclitaxel will be transported past the polymeric membrane through the nanopores, where the paclitaxel will be taken into cells of the vasculature at the target position,

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, for example, as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An angioplasty balloon comprising:
an inflatable balloon including a balloon surface; and
a coating on at least a portion of the balloon surface, wherein the coating includes an therapeutic agent, a first polymer, and a second polymer, the first polymer and the second polymer having different stereoisoisomeric forms;
wherein the first and the second polymer are capable of co-crystalizing to form a stereocomplex between the first and second polymers.

2. The angioplasty balloon of claim 1, wherein the therapeutic agent comprises one or more substances selected from the group consisting of: an antiproliferative agent, an antimitotic agent, an antiplatelet agent, an alkylating agent, an antimetabolite, a platinum coordination complex, a hormone, an anticoagulant, a fibrinolytic agent, an antimigratory agent, an antisecretory agent, an anti-inflammatory agent, indole and acetic acids, an immunosuppressive agent, an angiogenic agent, an angiotensin receptor blocker, a nitric oxide donor, an anti-sense oligonucleotide, a cell cycle inhibitor, a retinoid, a cyclin/CDK inhibitor, an HMG co-enzyme reductase inhibitor, and a protease inhibitor.

3. The angioplasty balloon of claim 1, wherein the therapeutic agent comprises paclitaxel.

4. The angioplasty balloon of claim 1, wherein the first polymer comprises a first nanoparticle and the second polymer comprises a second nanoparticle, wherein the therapeutic agent is at least partially contained within at least one of the first nanoparticle and the second nanoparticle.

5. The angioplasty balloon of claim 1, wherein the first polymer and second polymer each comprise a polylactide.

6. The angioplasty balloon of claim 1, wherein the first polymer comprises isotatic poly(L-lactide) and the second polymer comprises isotatic poly(D-lacticde).

7. A method for treating a target vascular portion of a subject, the method comprising:
providing an angioplasty balloon system including a balloon, the balloon carrying a first polymer, a second polymer, and at least one active agent on a surface of the balloon, the at least one active agent being at least partially contained within at least one of the first polymer and the second polymer, wherein the first polymer and the second polymer having different stereoisoisomeric forms that can co-crystalize to form a stereocomplex between the first and second polymers;
positioning the balloon proximate the target vascular portion;
expanding the balloon to engage the target vascular portion; thereby delivering at least a portion of the active agent to the target vascular portion; and
withdrawing the balloon from the subject.

8. The method of claim 7, further comprising forming a stereocomplex between the first and second polymers, whereby the formation of the stereocomplex releases the active agent from the at least one of the first polymer and second polymer.

9. The method of claim 8, wherein the stereocomplex is formed within one or more cells comprising the target vascular portion, whereby the formation of the stereocomplex releases a therapeutically effective amount of the therapeutic agent in the one or more cells.

10. The method of claim 7, wherein the first polymer comprises a first nanoparticle and the second polymer comprises a second nanoparticle, wherein the stereocomplex is formed by the first and the second polymers being driven together to form a third particle by stereocomplexation.

11. The method of claim 11, wherein the therapeutic agent comprises one or more substances selected from the group consisting of: an antiproliferative agent, an antimitotic agent, an antiplatelet agent, an alkylating agent, an antimetabolite, a platinum coordination complex, a hormone, an anticoagulant, a fibrinolytic agent, an antimigratory agent, an antisecretory agent, an anti-inflammatory agent, indole and acetic acids, an immunosuppressive agent, an angiogenic agent, an angiotensin receptor blocker, a nitric oxide donor, an anti-sense oligonucleotide, a cell cycle inhibitor, a retinoid, a cyclin/CDK inhibitor, an HMG co-enzyme reductase inhibitor, and a protease inhibitor.

12. The method of claim 11, wherein the therapeutic agent comprises paclitaxel.

13. The method of claim 11, wherein the first polymer and second polymer each comprise a polylactide.

14. The method of claim 11, wherein the first polymer comprises poly(L-lactide) and the second polymer particle comprises poly(D-lacticde).

15. An angioplasty balloon comprising:
an inflatable balloon including a balloon surface; and
a coating on at least a portion of the balloon surface, wherein the coating includes a polymersome enclosing a therapeutic agent.

16. The angioplasty balloon of claim 15, wherein the polymersome comprises a polymeric membrane and at least one DNA nanopore, the DNA nanopore providing a pathway through the polymeric membrane.

17. The angioplasty balloon of claim 16, wherein the pathway through the polymeric membrane defines a lumen diameter of about 2 nm.

18. The angioplasty balloon of claim 16, wherein the polymeric membrane comprises poly 2-(methacryloyloxy)ethyl phosphorylcholine-b-disisopropylamino) ethyl methacrylate.

19. The angioplasty balloon of claim 15, wherein the therapeutic agent comprises one or more substances selected from the group consisting of: an antiproliferative agent, an antimitotic agent, an antiplatelet agent, an alkylating agent, an antimetabolite, a platinum coordination complex, a hormone, an anticoagulant, a fibrinolytic agent, an antimigratory agent, an antisecretory agent, an anti-inflammatory agent, indole and acetic acids, an immunosuppressive agent, an angiogenic agent, an angiotensin receptor blocker, a nitric oxide donor, an anti-sense oligonucleotide, a cell cycle inhibitor, a retinoid, a cyclin/CDK inhibitor, an HMG co-enzyme reductase inhibitor, and a protease inhibitor.

20. The angioplasty balloon of claim 15, wherein the therapeutic agent comprises paclitaxel.
